(19) United States
(12) Patent Application Publication
(10) Pub. No.: US 2022/0332935 A1
(43) Pub. Date: Oct. 20, 2022

(54) Method for sterilization of formed articles made of acrylic-based polymers

(71) Applicants: Röhm GmbH, Darmstadt (DE); Roehm America LLC, Parsippany, NJ (US)

(72) Inventors: Kay Bernhard, Darmstadt (DE); Kierra Wright, Hamden, CT (US); Ralf Richter, Hanau (DE)

(73) Assignees: Röhm GmbH, Darmstadt (DE); Roehm America LLC, Parsippany, NJ (US)

(21) Appl. No.: 17/658,832

(22) Filed: Apr. 12, 2022

Related U.S. Application Data
(60) Provisional application No. 63/174,571, filed on Apr. 14, 2021.

(51) Int. Cl. C08L 33/08

(52) U.S. Cl. CPC C08L 33/08

(57) ABSTRACT

Formed articles, such as medical devices, can be made of an acrylic-based polymer material, which contains at least one polyalkyl(meth)acrylate. A method can be used for the UV disinfection and/or sterilization of the formed articles, including disinfection and/or sterilization of the inner surface of the formed article. The method for UV sterilization involves exposure of the outer surface of the formed article with UV radiation at a wavelength in the range of 260 nm to 300 nm, wherein the acrylic-based polymer material has a transmittance of at least 10%, averaged over the wavelength range from 260 nm to 300 nm, measured in accordance with ISO 13468-2 at a thickness of 3 mm.

### CROSS-REFERENCE TO RELATED APPLICATIONS

This application claims priority to U.S. Provisional Application No. 63/174,571, filed on Apr. 14, 2021, the content of which is hereby incorporated by reference in its entirety.

### BACKGROUND OF THE INVENTION

### Field of the Invention

The present invention relates to formed articles, such as medical devices, made of an acrylic-based polymer material, which comprises at least one polyalkyl(meth)acrylate, and a method for their UV disinfection and/or sterilization, including disinfection and/or sterilization of the inner surface of the formed article. The inventive method for UV sterilization comprises the exposure of the outer surface of the formed article with UV radiation at a wavelength in the range of 260 nm to 300 nm, wherein the acrylic-based polymer material has a transmittance of at least 10%, averaged over the wavelength range from 260 nm to 300 nm, and measured in accordance with ISO 13468-2 at a thickness of 3 mm.

### Description of Related Art

Medical grade acrylic-based polymer materials are commonly known in the state of the art and typically offer an excellent balance of optical and mechanical properties. These polymer materials are often transparent, have an excellent thermoplastic processability and can be advantageously used for production of medical devices, e.g. via injection molding. Typical applications of such devices include commonly known medical and medical diagnostic applications, such as intravenous and catheter accessories, blood handling devices, chest drainage units, respiratory ventilating devices etc.

Generally, medical devices, especially disposables devices intended for intravenous application (e.g. IV-Sets), need to be sterilized before use. This is usually done by the manufacturer, so the devices are ready to use. Medical devices made from acrylic-based polymer materials are typically sterilized using chemical sterilization agents, such as ethylene oxide, or using gamma rays, e-beam or X-radiation. As a rule, only a small share of about 10% is sterilized by X-ray or electron beam exposure, most of such medical devices are sterilized by gamma radiation or ethylene oxide treatment (each about 45%). Both procedures are well established in the medical industry and considered state of the art, but they involve a lot of disadvantages.

In the case of sterilization using gamma radiation, radioactive radiation and substances, mainly Co-60 (t_{1/2}=5.27 a) need to be handled. That requires a lot of investment and very strict processes. Facilities for gamma radiation sterilization need to be rather large to compensate for these investments via economy of scales. Lately a shortage of Co-60 has been reported. In the case of sterilization using ethylene oxide, a similar level of precautions is needed, because ethylene oxide is extremely flammable and carcinogenic. This process also needs certain requirements in packaging since the ethylene oxide needs to reach the medical devices directly. Facilities also need to be rather large to compensate for these investments via economy of scales. Sometimes ethylene oxide sterilization sites have to be closed because of too high ethylene oxide exposure.

The sterilization effect of ultraviolet (UV) irradiation has been known since the latter half of the 19th century, and in recent years, the use of UV irradiation has been widely accepted in the field of water and air purification, as well as in disinfection and sterilization in food processing or of medical equipment. For example, UV radiation is used for disinfection in hospitals, nurseries, operating rooms, cafeterias and to sterilize vaccines, serums, toxins, municipal waste, and drinking waters. Generally, UV light is separated into the categories of UV-A (typically of wavelength range from 315 to 400 nm), UV-B (typically of wavelength range from 280 to 315 nm), and UV-C (typically of wavelength range from 200 to 280 nm).

Specifically, UV radiation does not kill microorganisms directly, but damages the genetic material, the deoxyribonucleic acid (DNA), of the microorganisms, e.g. pathogens. This inhibits the cell division ability and effectively kills the microorganism. Generally, UV-C radiation have been shown to initiate a photoreaction between adjacent pyrimidine bases, such as cytosine and thymine, which exhibit conjugated double bonds and as such absorb UV light. The germicidal effectiveness of UV-C radiation peaks at about 260-265 nm and typically corresponds to the maximum UV absorption by bacterial DNA. In particular, the optimum wavelength for germicidal effectiveness varies between different microorganism species. Generally, UV radiation in UV-B range only have a small contribution to the inactivation of microorganisms (W. J. Kowalski, “UVGI Disinfection Theory”, in Ultraviolet Germicidal Irradiation Handbook, pp. 17-25, July 2009, DOI: 10.1007/978-3-642-01999-9_2).

Generally, the photoreaction between adjacent thymine or cytosine bases proceeds at an exceedingly rapid rate, wherein one common reaction is the formation of a cyclo-butane ring between the two pyrimidines. The inactivation of specific genes via punctual mutations is one of the mechanisms of UV-induced genetic damage that effect inhibition of cell replication and cell death (W. J. Kowalski, “UVGI Disinfection Theory”, in Ultraviolet Germicidal Irradiation Handbook, pp. 17-25, July 2009, DOI: 10.1007/978-3-642-01999-9_2).

UV-C radiation in the range of 220-280 nm is widely used in industry for disinfection of liquids, e.g. water, and surfaces. Since water is UV transparent at 220 nm, this is a very common wavelength for disinfection of well water. Mercury UV lamps, which are widely used for disinfection and sterilization, emit at 254 nm. Further, DNA specifically has a high absorbency at said wavelength. Thus, wavelength of 254 nm has been and still is widely used in prior art germicidal devices. Indeed, with the development of UV-LEDs the range of 260-280 nm became accessible.

It is well-known that most of common transparent polymeric materials, such as polycarbonates (PC), polyethylene terephthalates (PET), have only a limited transmittance of UV light, in particular have a low or no transmittance below 280 nm. Therefore, often complex parts cannot be UV sterilized since the UV radiation does not reach inner surfaces, voids, and cavities. Polymers containing aromatic moieties like polystyrene and polyethylene terephthalates, are intrinsically not UV transparent. Other polymers, like PVC or PMMA, often comprises at least some UV absorbing additives, such as softeners (phthalates) or stabilizer. Pure poly(methyl)methacrylate (PMMA) shows a good transmittance in the UV-A range (320-400 nm) and some transmittance in UV-B and UV-C. In practice, however, certain processing additives, such as UV stabilizers, may lead to reduced UV transmittance of PMMA materials.

The document JP63095402 A describes a core-sheath type plastic optical fiber having excellent translucency near-ultraviolet wavelengths about 400 nm, which is necessary for animal and plant growth and fungal growth or sterilization. The core component is made from a polymer comprising mainly methyl methacrylate units, wherein the content of remaining methyl methacrylate monomer in the polymer of the core component is <=4,000 ppm.

The document U.S. Pat. No. 7,834,328 B2 describes a method and apparatus for sterilizing access sites, such as attachment points for various therapeutic and diagnostic medical devices. More particularly, U.S. Pat. No. 7,834,328 B2 concerns a sterilization apparatus which includes a substantially UV-C transparent closure cap for closing the access site and an irradiating apparatus for irradiating the closure cap with UV-C radiation. Typically, said closure can be built up entirely from UV-C transparent materials or comprises a hollow, substantially UV-C opaque body; and a substantially transparent part. The UV transparent material is for example selected from quartz glass or polyethylene. Sterilizing is carried out using UV-C radiation in the wavelength range of 200 to 280 nm.

The document JP5938408B2 describes a vascular access device including a fluid chamber having a lumen, an inner surface, an outer surface, and an irradiation window, wherein the irradiation window is made of a UV-C transmissive material so that pathogens in the lumen can be irradiated with UV-C radiation of 250 to 265 nm. For example, the illumination window is made of optical quartz or fluorinated polymer material.

Further, it is known from WO 2018/033502 A1 to use an optical element made of a polymeric material selected from polyalkyl(meth)acrylate (PMMA) or poly(meth)acrylalkylimide (PMMI) in an UV light engine. It is described, that pure poly(methyl)methacrylate (PMMA) shows a good transmittance in the UVA range (320-400 nm). The yellowing of PMMA during UV radiation exposure should be reduced by the selection of certain stabilizers and a specific employed ultraviolet light source having an emission peak wavelength between 350 nm and 400 nm.

Several known medical grade acrylic-based polymer compositions comprise copolymers of alkyl(meth)acrylates, aromatic vinyl monomer and unsaturated carboxylic acid anhydride (e.g. maleic anhydride), typically a particulate impact modifier and/or a further polymeric component, such as a styrene-acrylonitrile copolymer (SAN). For example, the documents WO 2020/126722 A1, WO 2008/148595 A1, and JP H02-272050 A2 describe such transparent polymer composition having an increased chemical resistance. Such medical grade acrylic-based polymer compositions already have a good chemical resistance to alcohols, which are commonly used as disinfectants for medical devices, but they tend to become turbid and get cracks upon a long-term exposure to isopropanol-water mixtures. Further, such known medical grade acrylic-based polymer compositions are not suitable for UV sterilization, in particular not for UV sterilization of their inner surface, due to their low transmittance for UV-C radiation.

Thus, there is the need for novel, easy and effective methods for sterilization of medical devices, in particular disposable medical devices, avoiding the impairment of optical and mechanical properties of said medical devices.

An object of the invention is to provide formed articles, such as medical devices, which exhibit excellent mechanical and optical properties, e.g. high transparency, and which can be easily and effectively be sterilized using UV radiation, including the inner surface of the formed article. Another object is directed to the corresponding easy and cost-effective method for UV sterilization of the inner surface of said formed articles. Further, an object of the present invention is to provide formed articles, such as disposable medical devices, which can be produced easily and cost-effectively via a thermal molding process, e.g. via injection molding.

### SUMMARY OF THE INVENTION

Generally, pure polymethylmethacrylate (PMMA) or some copolymers of methyl methacrylate, which are completely or nearly free of UV absorbing additives, provide a certain transparency to UV-C light at 265 nm. Typically, up to 25% of such UV-C light pass a pure PMMA material having 3 mm thickness and stay effective towards germs. Radiation of higher wavelength up to 280 nm in UV-B and UV-A ranges, is less effective, but the transmittance of PMMA is much higher in said range. Therefore, this ranges of transmitting UV radiation, preferably from 280 nm to 300 nm, can be used for sterilization and/or disinfection as well.

It was surprisingly found, that these UV transmittance of specific PMMA material is high enough to provide an effective and safe sterilization and/or disinfection of the inner surface of arbitrary formed articles made of said PMMA material, when UV radiation in the range of 260 to 300 nm is used. Further, PMMA can be combined with other UV-C transparent polymers like silicone or UV-C transparent PVC-tubing (i.e. phthalate free PVC, e.g. non-DEHP-PVC-tubing).

Particularly, that specific acryl-based polymer materials, typically PMMA homo- and copolymers in combination with certain additives and/or stabilizers, exhibit a good transmittance in the relevant UV range, i.e. at least 10%, averaged or summarized over the range of 260 to 300 nm, preferably of 260 to 280 nm, most preferably at 265 nm. Furthermore, other important optical and mechanical properties of the specific acrylic-based polymer material and formed articles made thereof are good to excellent as well. In particular the acrylic-based polymer material exhibits low haze and high transparency, excellent biocompatibility, good scratch and chemical resistance, high heat distortion resistance, high modulus of elasticity and/or high Vicat softening temperature.

Therefore, these acrylic-based polymer materials can be advantageously used as material for formed articles, that are subject to a UV sterilization process, such as medical devices, e.g. disposable medical devices, or various containers and/or packaging used in food industry, pharmaceutical industry or cosmetic industry.

Advantageously, it is possible to achieve a sterilization and/or disinfection of the whole surface of the formed article, including the inner surface of the formed article, for example the inner surface of openings and/or cavities of the formed article.

The present invention is directed to a method for UV sterilization of a surface of a formed article, such as a medical device or a food container, including sterilization of the inner surface of the formed article, comprising the steps:
a. providing a formed article, wherein at least one part of said formed article is made from (consists of) an acrylic-based polymer material, which comprises at least 50 wt.-%, preferably at least 60 wt.-%, more preferably at least 70 wt.-%, based on the acrylic-based polymer material, of at least one alkyl(meth)acrylate polymer A;
b. exposure of the outer surface of the formed article with UV radiation at a wavelength in the range of 260 to 300 nm, preferably 260 nm to 280 nm;
wherein the acrylic-based polymer material has a transmittance of at least 10%, preferably at least 15%, more preferably at least 20%, also preferably at least 25%, averaged over the wavelength range from 260 nm to 300 nm, preferably from 260 to 280 nm, and measured in accordance with ISO 13468-2 at a thickness of 3 mm.

A further aspect of the present invention relates to a formed article, in particular for use as medical device, in food, cosmetic and/or pharmaceutical industry, comprising at least one part made of the acrylic-based polymer material. Importantly, the inventive formed articles exhibit an advantageous balance of properties, including high transmittance in the relevant UV range needed for sterilization as well as a number of further advantageous properties, such as excellent biocompatibility, low haze (i.e. high transparency in the VIS range), excellent processability, good mechanical properties, such as good scratch resistance, high heat distortion resistance, high modulus of elasticity and/or high Vicat softening temperature.

The acrylic-based polymer material can be prepared and processed in a relatively simple manner and is particularly suitable for the production of articles having a complex geometrical shape, using a commonly known thermal molding process, in particular injection molding. Hence, in this further aspect, the present invention relates to a process for producing the formed article from the acrylic-based polymer material, preferably via injection molding.

Finally, a further aspect of the present invention is directed to the use of the inventive formed article in a method for UV sterilization, wherein UV radiation at a wavelength in the range of 260 nm to 300 nm, preferably 260 nm to 280 nm, is utilized.

The invention also includes the following embodiments:
1. Method for UV sterilization of a surface of a formed article, including sterilization of the inner surface of the formed article, comprising the steps:
   a. providing a formed article, wherein at least one part of said formed article is made from an acrylic-based polymer material, which comprises at least 50 wt.-%, based on the acrylic-based polymer material, of at least one alkyl (meth)acrylate polymer A;
   b. exposure of the outer surface of the formed article with UV radiation at a wavelength in the range of 260 nm to 300 nm;
   wherein the acrylic-based polymer material has a transmittance of at least 10%, averaged over the wavelength range from 260 nm to 300 nm, and measured in accordance with ISO 13468-2 at a thickness of 3 mm.
2. Method according to Embodiment 1, wherein the acrylic-based polymer material has a transmittance of at least 8%, preferably at least 10%, also preferably at least 15%, more preferably at least 20%, at a wavelength of 265 nm, measured in accordance with ISO 13468-2 at a thickness of 3 mm.
3. Method according to Embodiment 1 or 2, wherein the acrylic-based polymer material comprises, based on the total weight of the acrylic-based polymer material:
   A. 50.0 to 100.0 wt.-%, preferably 54.0 to 93.0 wt.-%, of at least one alkyl(meth)acrylate polymer A;
   B. 0.0 to 40.0 wt.-%, preferably 5.0 to 35.0 wt.-%, of at least one impact modifier B, preferably selected from particulate multiphase graft copolymers;
   C. 0.0 to 50.0 wt.-%, preferably 1.0 to 40.0 wt.-%, of at least one polymeric component C, different from A and B, preferably selected from polyvinylidene fluoride (PVDF) and/or polyethylene glycols (PEG) having a molecular weight of at least 10,000 g/mol;
   D. 0.0 to 20.0 wt.-%, preferably 1.0 to 10.0 wt.-%, of at least one plasticizer D, and
   E. 0.0 to 10.0 wt.-%, preferably 0.0 to 5.0 wt.-%, of at least one further component E, preferably selected from additives, auxiliaries and/or fillers.
4. Method according to any of Embodiments 1 to 3, wherein the acrylic-based polymer material comprises:
   at least 50 wt.-%, preferably at least 60 wt.-%, more preferably at least 70 wt.-%, based on the acrylic-based polymer material, of at least one alkyl (meth)acrylate polymer A,
   at least one impact-modifier B, selected from particulate multiphase graft copolymers, which comprises an elastomeric phase built up from crosslinked C1-C10 alkyl acrylate, preferably n-butyl acrylate, and a hard, outer shell comprising at least one C1-C10 alkyl (meth)acrylate, preferably methyl methacrylate; and
   at least one polymeric component C selected from polyvinylidene difluoride (PVDF),
   wherein the ratio of the components A:C is in the range of 3:1 to 4:1, preferably of 3.3:1 to 3.5:1, more preferably about 3.4:1.
5. Method according to any of Embodiments 1 to 4, wherein the alkyl(meth)acrylate polymer A comprises, based on the total weight of the alkyl(meth)acrylate polymer A:
   70.0 to 100.0 wt.-%, preferably from 80.0 to 100.0 wt.-%, more preferably from 90.0 to 99.9 wt.-%, of at least one alkyl methacrylate monomer having from 1 to 20, preferably from 1 to 12, more preferably from 1 to 8, most preferably from 1 to 4, carbon atoms in the alkyl radical, and
   0.0 to 30.0 wt.-%, preferably from 0.0 to 20.0 wt.-%, more preferably from 0.1 to 10.0 wt.-%, of at least one alkyl acrylate monomer, having from 1 to 20, preferably from 1 to 12, more preferably from 1 to 8, in particular from 1 to 4, carbon atoms in the alkyl radical.
6. Method according to any of Embodiments 1 to 5, wherein the formed article is selected from medical devices; containers and/or packages used for cosmetic or pharmaceutical products or used in food industry.
7. Method according to any of Embodiments 1 to 6, wherein the UV radiation at a wavelength in the range of 260 to 300 nm is provided by one or more appropriate UV radiation sources, selected from UV light emitting diodes (UV-LEDs), wherein at least a significant amount of emission spectrum of the UV source, is in the range of 260 nm to 300 nm, preferably 260 to 280 nm.
8. Method according to any of Embodiments 1 to 7, wherein the exposure of the outer surface of the formed article with UV radiation is carried out in such way that a UV dose in the range of 1 to 10,000 mJ/cm² is effected.
9. Method according to any of Embodiments 1 to 8, wherein the method for UV sterilization inhibit the growth of one or more microorganisms of genus selected from Escherichia, Salmonella, Listeria, Aspergillus, Bacillus, Cryptosporidium, Clostridium, Streptomyces, Aeromonas, Candida, Helicobacter, Klebsiella, Legionella, Listeria, Pseudomonas, Staphylococcus, Streptococcus, Lactobacillus, Bifidobacterium, Oenococcus, and Saccharomycodes.
10. Method according to any of Embodiments 1 to 9, wherein the formed article is surrounded by a protective packaging during the UV exposure step b, wherein the protective packaging is made of an UV-transmissive polymer material, preferably selected from polyethylene, polypropylene, polyvinylchloride and acrylic-based polymer material as described in any of Embodiments 1 to 9.
11. Formed article for use in the method for UV sterilization according to any of Embodiments 1 to 10, wherein at least one part of said formed article is made from an acrylic-based polymer material, which comprises at least 50 wt.-%, based on the acrylic-based polymer material, of at least one alkyl(meth)acrylate polymer; and wherein the acrylic-based polymer material has a transmittance of at least 10% averaged over the wavelength range from 260 nm to 300 nm, and measured in accordance with ISO 13468-2 at a thickness of 3 mm.
12. Formed article according to Embodiment 11, wherein the formed article is selected from medical devices; containers and/or packages used for cosmetic or pharmaceutical products or used in food industry.
13. Formed article according to Embodiment 11 or 12, wherein the formed article is a medical device selected from medical diagnostic devices, intravenous and catheter accessory, blood handling devices, chest drainage units, respiratory ventilating devices, medical filter housings, permanent device housings, tubes, connectors, fittings, and cuvettes.
14. Process for producing a formed article according to any of Embodiments 11 to 13, wherein the process comprises a step, selected from injection molding, blow molding and thermoforming, of the acrylic-based polymer material.
15. Use of a formed article according to any of Embodiments 11 to 13 in a method for UV sterilization, wherein UV radiation at a wavelength in the range of 260 nm to 300 nm is utilized.

### BRIEF DESCRIPTION OF THE DRAWINGS

FIG. 1 shows the results of the UV sterilization test, showing the bacterial growth on petri dishes after UV exposure and incubation.

FIG. 2 shows the transmission spectra of acrylic-based polymer materials as described in the examples, as a function of the wavelength in a range from 250 to 900 nm.

FIG. 3 shows the transmission spectra of acrylic-based polymer materials as described in the examples, as a function of the wavelength in a range from 240 to 400 nm.

### DETAILED DESCRIPTION OF THE INVENTION

Often sterilization is understood as a process of complete elimination or destruction of all forms of microorganisms, including both vegetative and spore forms, preferably from inanimate objects, wherein sterilization may be carried out by various physical and chemical methods. Often sterilization is achieved by dry or moist heat, irradiation or gassing with ethylene oxide, formaldehyde or vaporized hydrogen peroxide (VHP). Often disinfection is understood as a process of reduction of the number of microorganisms by killing or irreversibly inactivating vegetative forms of microorganisms, e.g. pathogenic microorganisms, except bacterial spores, from inanimate objects and to interrupt infection chains. Often decontamination is defined as a process of removal of pathogenic microorganisms from inanimate objects, so that they are safe to handle (S. Mohapatra, Sterilization and Disinfection, Essentials of Neuroanesthesia. 2017: 929-944, Clinical and Laboratory Standards Institute).

In terms of the present invention the term “sterilization” or “method for sterilization” includes sterilization, disinfection and/or decontamination. In particular, the term “sterilization” or “method for sterilization” according to the invention encompasses the reduction of the number of microorganisms by killing and/or irreversibly inactivating growth of microorganisms. Preferably, the inventive process results in a colony forming units (CFU) log reduction on the inner surface of the formed article of at least 1, more preferably at least 2, more preferably at least 3, more preferably at least 4, also preferably at least 5, even more preferably of at least 6, of relevant microorganisms, preferably of vegetative forms of microorganisms.

In terms of the present invention “inner surface of the formed article” means the surface of the formed article, which is not directly exposed to the UV radiation and/or which is opposite to the surface exposed to the UV radiation. Typically, the inner surface may include openings, cavities and/or pores, which can typically not or only with high difficulties be exposed to UV radiation directly.

As used herein, the term “ultraviolet radiation” or “UV radiation” refers to radiation having a wavelength or wavelengths in the range from 200 nm to 400 nanometers (nm). If a range is specified, such as 260 nm to 300 nm, the range specified, unless otherwise indicated, means radiation including emission at one or more wavelengths within this specified range.

As used herein, the abbreviation “UV-A” refers to ultraviolet radiation in the range of ≥315 nm to ≤400 nm. As used herein, the abbreviation “UV-B” refers to ultraviolet radiation in the range of >280 nm to <315 nm. As used herein, the abbreviation “UV-C” refers to ultraviolet radiation in the range of a ≥200 to ≤280 nm.

As used herein, the term “ultraviolet and visible radiation” or “UV/Vis radiation” refers to radiation having a wavelength or wavelengths in the range from 200 nm to 800 nm, preferably 240 to 780 nm.

In terms of the present invention the term “polymer or copolymer comprising or consisting of monomer(s)” is understood in that the polymer or copolymer comprises or consists of said monomer unit(s). As a skilled person understands said polymers or copolymers are obtained by polymerisation of the referred monomers, wherein at least one of the unsaturated groups of the monomers is polymerized, preferably radically polymerized. As a skilled person understands such polymer or copolymer may also include groups resulting from radical initiators and/or molecular weight regulators. In case, that the polymer obtained after said polymerization comprises unreacted monomers, which are not incorporated in the polymer chain, this is referred to as residual monomer(s).

The term “polyalkyl(meth)acrylate” as used herein refer to homo- and copolymers comprising alkyl(meth)acrylate units.

Acrylic-Based Polymer Material

The acrylic-based polymer material comprises (also referred to as polymer material in the following) at least 50 wt.-%, preferably at least 60 wt.-%, more preferably at least 70 wt.-%, also preferably at least 80 wt.-%, based on the total polymer material, of at least one alkyl(meth)acrylate polymer A, optionally one or more impact modifiers B, optional one or more polymeric components C, optional one or more plasticizers D, and optionally one or more further components E, preferably selected from additives, auxiliaries and/or fillers.

In a preferred embodiment the acrylic-based polymer material comprises, based on the total weight of the acrylic-based polymer material:
A. 50.0 to 100.0 wt.-%, preferably 54.0 to 93.0 wt.-%, of at least one alkyl(meth)acrylate polymer A;
B. 0.0 to 40.0 wt.-%, preferably 5.0 to 35.0 wt.-%, of at least one impact modifier B, preferably selected from particulate multiphase graft copolymers, more preferably selected from particulate multiphase graft copolymers, which comprise a elastomeric phase built up from crosslinked C1-C10 alkyl acrylate, preferably n-butyl acrylate, and a hard, outer shell comprising at least one C1-C10 alkyl (meth)acrylate, preferably methyl methacrylate;
C. 0.0 to 50.0 wt.-%, preferably 1.0 to 40.0 wt.-%, of at least one polymeric component C, different from A and B, preferably selected from polyvinylidene fluoride (PVDF) and/or polyethylene glycols (PEG) having a molecular weight of at least 10,000 g/mol;
D. 0.0 to 20.0 wt.-%, preferably 1.0 to 10.0 wt.-%, of at least one plasticizer D, and
E. 0.0 to 10.0 wt.-%, preferably 0.0 to 5.0 wt.-%, of at least one further component E, preferably selected from additives, auxiliaries and/or fillers.

In another embodiment, the acrylic-based polymer material comprises, based on the total weight of the acrylic-based polymer material:
A. 80.00 to 99.99 wt.-%, preferably 85.00 to 99.90 wt.-%, more preferably 90 to 99.00 wt.-%, of at least one alkyl(meth)acrylate polymer A;
D. 0.01 to 20.00 wt.-%, preferably 0.10 to 15.00 wt.-%, more preferably 1.00 to 10.00 wt.-%, of at least one plasticizer D, preferably selected from of at least one polyethylene glycol having a molecular weight from 500 to <10 000 g/mol; and
E. 0.00 to 10.00 wt.-%, preferably 0.00 to 5.00 wt.-%, of at least one further component E, preferably selected from additives, auxiliaries and/or fillers.

In another preferred embodiment the acrylic-based polymer material comprises, based on the total weight of the acrylic-based polymer material:
A. 50.0 to 99.0 wt.-%, preferably 69.0 to 89.0 wt.-%, of at least one alkyl(meth)acrylate polymer A;
C. 1.0 to 40.0 wt.-%, preferably 10.0 to 30.0 wt.-%, of at least one polymeric component C, preferably selected from polyvinylidene fluoride (PVDF) and/or polyethylene glycols (PEG) having a molecular weight of at least 10,000 g/mol;
D. 0.0 to 20.0 wt.-%, preferably 1.0 to 10.0 wt.-%, of at least one plasticizer D, and
E. 0.0 to 10.0 wt.-%, preferably 0.0 to 5.0 wt.-%, of at least one further component E, preferably selected from additives, auxiliaries and/or fillers.

According to the present invention the acrylic-based polymer material has a transmittance of at least 10%, preferably at least 15%, more preferably at least 20%, also preferably at least 25%, most preferably at least 30%, averaged over the wavelength range from 260 nm to 300 nm, preferably from 260 to 280 nm, and measured in accordance with ISO 13468-2 at a thickness of 3 mm.

Further preferably, the acrylic-based polymer material has a transmittance of at least 8%, preferably at least 10%, more preferably at least 15%, at a wavelength of 265 nm, measured in accordance with ISO 13468-2 at a thickness of 3 mm.

Further preferably, the polymeric material has a total transmittance of at least 10%, preferably at least 20%, more preferably at least 30%, integrated over the wavelength range from 260 to 300 nm, and measured in accordance with ISO 13468-2 at a thickness of 3 mm.

Generally, the transmittance of the acrylic-based polymer material at the wavelength of 265 nm or in the range of 260 to 300 nm can be measured using an instrument as defined in the standard ISO 13468-2 which is suitable for measurements in said wavelength range (e.g. a Varian Cary 5000). Generally, the transmittance of a material is defined as the ratio of transmitted optical power to the incident optical power for a given thickness, wherein the light resulting from directed transmission as well as from diffuse transmission is included.

Preferably, the acrylic-based polymer material and/or the formed article made thereof are transparent in the visible range of electromagnetic spectrum. In terms of the present invention. “a transparent polymer materiar” or “transparent formed article” means that the polymer material or the formed article has a haze according to standard ASTM D1003 of less than 50%, preferably less than 40%, more preferably less than 30%, most preferably less than 20%, measured at 23° C. on an injection molded specimen having a thickness of 3 mm.

The acrylic-based polymer material can be prepared by dry blending the components, which may be present as powder, particles or preferably pellets. The acrylic-based polymer material can also be prepared by mixing the components either at the same time or successively into the melt of the alkyl(meth)acrylate polymer A, optional in the melt of alkyl(meth)acrylate polymer A and optional polymeric component C. The acrylic-based polymer material can also be prepared by melting and mixing the individual components in the molten state or by melting dry premixes of the individual components to give a ready-to-use molding material. This can be effected, for example, in single-screw or twin-screw extruders. The extrudate obtained can then be granulated. Customary additives, auxiliaries and/or fillers can be directly admixed or added later by end users as required.

In particular, the acrylic-based polymer material represents a thermoplastic molding composition, that can be processed via commonly known thermal molding processes, e.g. via injection molding, to produce formed articles having various arbitrary shape.

In particular, the present invention is directed to a method for UV sterilization of a formed article, wherein the formed article is selected from medical devices, containers and/or packages used for cosmetic or pharmaceutical products or used in food industry, for example in production and packaging of beverages, meat products, or dairy products.

Alkyl(Meth)Acrylate Polymer A

Preferably, the acrylic-based polymer material comprises from 50.0 to 100.0 wt.-%, preferably 60.0 to 99.9 wt.-%, more preferably 70.0 to 95.0 wt.-%, based on the total polymer material, of the alkyl(meth)acrylate polymer A.

The alkyl(meth)acrylate polymer A is preferably composed of one or more thermoplastic polyalkyl(meth)acrylates.

The term “alkyl(meth)acrylates” as used herein may stand for a single alkyl(meth)acrylate or as a mixture of different alkyl(meth)acrylates. Respectively the term “polyalkyl(meth)acrylate” as used herein may stand for polymers and copolymers made from single alkyl(meth)acrylate or from a mixture of different alkyl(meth)acrylates and optionally other comonomers. The term “(meth)acrylate” as used herein refers to methacrylates, e.g. methyl methacrylate, ethyl methacrylate, as well as acrylates, e.g. methyl acrylate, ethyl acrylate. etc. and mixtures thereof.

For the purposes of the present invention, particular preference is given to C1-C18-alkyl (meth)acrylates, advantageously C1-C10-alkyl(meth)acrylates, in particular C1-C4-alkyl (meth)acrylates. Preferred alkyl methacrylates encompass methyl methacrylate (MMA), ethyl methacrylate, propyl methacrylate, isopropyl methacrylate, n-butyl methacrylate, isobutyl methacrylate, tert-butyl methacrylate, pentyl methacrylate, hexyl methacrylate, heptyl methacrylate, octyl methacrylate, isooctyl methacrylate, and ethylhexyl methacrylate, nonyl methacrylate, decyl methacrylate, and also cycloalkyl methacrylates, for example cyclohexyl methacrylate, isobornyl methacrylate or ethylcyclohexyl methacrylate. Use of methyl methacrylate is particularly preferred.

In a preferred embodiment the alkyl(meth)acrylate polymer A comprises (preferably consists of), based on the total weight of the alkyl(meth)acrylate polymer A:
70.0 to 100.0 wt.-%, preferably from 80.0 to 100.0 wt.-%, more preferably from 90.0 to 99.9 wt.-%, of at least one alkyl methacrylate monomer (respectively repeat unit) having from 1 to 20, preferably from 1 to 12, more preferably from 1 to 8, most preferably from 1 to 4, carbon atoms in the alkyl radical, most preferably methyl methacrylate, and
0.0 to 30.0 wt.-%, preferably from 0.0 to 20.0 wt.-%, more preferably from 0.1 to 10.0 wt.-%, of at least one alkyl acrylate monomer (respectively repeat unit), having from 1 to 20, preferably from 1 to 12, more preferably from 1 to 8, in particular preferably from 1 to 4, carbon atoms in the alkyl radical, most preferably methyl acrylate and/or ethyl acrylate.

Particularly, the alkyl(meth)acrylate polymer A comprises, based on its total weight, at least 50.0 wt.-%, advantageously at least 60.0 wt.-%, preferably at least 75.0 wt.-%, in particular at least 85.0 wt.-%, methyl methacrylate.

In a particularly preferred embodiment, the polyalkyl(meth)acrylate comprise 80.0 to 100.0 wt.-%, preferably 90.0 to 100.0 wt.-%, more preferably 95.0 to 100.0 wt.-% methyl methacrylate (MMA), and 0.0 to 20.0 wt.-%, preferably 0.0 to 10.0 wt.-%, more preferably 0.0 to 5.0 wt.-% of an alkyl(meth)acrylate other than MMA, each based on the weight of the polyalkyl(meth)acrylates. The alkyl(meth)acrylate other than MMA can be selected from substantially any of the preferred alkylmethacrylates and alkyl acrylates listed above, preferably from methyl acrylate, ethyl acrylate, butylacrylate and butylmethacrylate.

In a preferred embodiment the polyalkyl(meth)acrylate is a copolymer comprising from 80 to 99 wt.-% of methyl methacrylate and from 1 to 20 wt.-% of C1-C10-alkyl acrylate units, preferably methyl acrylate and/or ethyl acrylate, based in each case on the weight of the copolymer. For instance, the alkyl(meth)acrylates may consist of MMA and ethyl acrylate or of MMA and methyl acrylate. In yet a further preferred embodiment, the alkyl(meth)acrylates solely consist of MMA.

Typically, the alkyl(meth)acrylate polymer A has a number-average molar mass in the range from 1000 to 100,000,000 g/mol, preferably in the range from 10,000 to 1,000,000 g/mol, preferably in the range from 50,000 to 500,000 g/mol. This molar mass may be determined by gel permeation chromatography, for example, with calibration based on polystyrene.

Preferably, the acrylic-based polymer material is free of polymers and copolymers comprising mono vinyl aromatic monomers, such as styrene.

Impact-Modifier B

In a preferred embodiment, the acrylic-based polymer material comprises 0.0 to 40.0 wt.-%, preferably 0.0 to 35.0 wt.-%, also preferably 5.0 to 30.0 wt.-%, based on the total polymer material, of at least one impact modifier as component B. Typically, impact strength, craze resistance and/or chemical resistance can be improved by the addition of one or more impact modifiers. Preferably, the optional impact modifier B is selected, so that it does not significantly lower the UV transmittance of the acrylic-based polymer material in the desired range, e.g. from 260 to 300 nm, preferably from 260 to 280 nm, more particularly at 265 nm.

Preferably, the optional impact-modifier B is selected from particulate multiphase graft copolymers (also referred to as graft copolymer in the following). Typically, the term “particulate multiphase graft copolymer” is directed to crosslinked graft copolymers, which may have a core-shell structure including at least one core and at least one shell. For example, the particulate multiphase graft copolymer may be formed by crosslinked particles having core-shell structure or core-shell-shell structure.

Typically, the particles have an average particle diameter from 20 nm to 500 nm, preferably from 50 nm to 450 nm, more preferably from 100 nm to 400 nm. Average particle diameter can be determined by a method known to a skilled person. e.g. by photon correlation spectroscopy according to DIN ISO 13321:1996 or via transmission electron microscopy.

Typically, the particulate impact modifier B is present in the acrylic-based polymer material as a dispersed phase, i.e. dispersed in a polymer matrix which is formed by the alkyl(meth)acrylate polymer A and optionally components C, D and/or E.

Preferably, the optional impact-modifier B is selected from particulate multiphase graft copolymers, which comprises a core and at least one shell, wherein typically the outer shell represents a hard phase comprising at least one alkyl(meth)acrylate. Typically, the particulate multiphase graft copolymer comprises an elastomeric core or at least one elastomeric intermediate layer. In terms of the present invention an elastomeric core or an elastomeric layer (also referred to as soft core or soft layer) means a core or layer composed of a polymer or of a polymer composition having a glass transition temperature T_{g}<20° C., preferably T_{g}<0° C.

Generally, the glass transition temperature T_{g} of a polymer or of a phase of a polymer, e.g. of a particulate multiphase graft copolymer, can be determined in a known manner by differential scanning calorimetry (DSC). The glass transition temperature T_{g} may also be calculated as an approximation by means of the Fox equation.

The impact modifier B may be selected from known particulate impact modifiers, for example based on a crosslinked poly(meth)acrylate elastomeric phase (core or intermediate layer). Typically, the particulate multiphase graft copolymer exhibits a hard, outer shell comprising (preferably consisting of) at least one alkyl(meth)acrylate and optionally other comonomers, preferably non UV-absorbing monomers. Preferably, the hard, outer shell exhibit the same or similar refraction index as the matrix formed by the acrylic-based polymer material.

Preferably, the impact-modifier B is free of any components, that significantly reduce the transmittance at the desired UV wavelength range, e.g. from 260 to 300 nm, preferably from 260 to 280 nm, more particularly at 265 nm. In particular, the impact-modifier is free of polymers and copolymers comprising mono vinyl aromatic monomers, such as styrene.

In one preferred embodiment, the optional impact modifier B is a particulate multiphase graft copolymer based on an elastomeric crosslinked alkyl(meth)acrylate core, preferably a crosslinked C1-C10 alkyl acrylate core, more preferably a crosslinked n-butyl acrylate core. Preferably, the particulate multiphase graft copolymer comprises a soft, elastomeric core and a hard, non-elastomeric outer phase, which is produced in the presence of the core, typically via graft emulsion polymerization (core-shell graft copolymer).

In another preferred embodiment the particulate multiphase graft copolymer comprises a hard, non-elastomeric core; a soft, elastomeric intermediate shell, which is produced in the presence of the core, typically via graft emulsion polymerization, and a hard, non-elastomeric outer shell, which is produced in the presence of the intermediate core-shell particles, typically via graft emulsion polymerization (core-shell-shell graft copolymers).

Preferably, the particulate multiphase graft copolymer comprises an elastomeric phase, which is for example built up from crosslinked C1-C10 alkyl acrylate, preferably n-butyl acrylate; and a hard, outer shell, that is for example built up from non-crosslinked C1-C10 alkyl(meth)acrylate, e.g. methyl methacrylate. Typically, said graft copolymers are produced as described in EP 1 332 166 B1, WO 02/20634 or EP 0 522 351.

Preferably, the outer shell of the particulate multiphase graft copolymer is a hard phase comprising at least 80 wt.-%, based on the outer shell, at least one C1-C6 alkyl methacrylate, preferably at least 80 wt.-%, based on the outer shell, of methyl methacrylate. Also preferably, the outer shell is a hard phase comprising 80 to 100 wt.-% methyl methacrylate and 0 to 20 wt.-% of at least one C1-C10 alkyl acrylate, such as methyl acrylate, ethyl acrylate, and n-butyl acrylate, preferably n-butyl acrylate.

According to a preferred embodiment, the impact modifier B is selected from particulate multiphase graft copolymers including an elastomeric crosslinked alkyl(meth)acrylate phase, wherein the particulate multiphase graft copolymer comprises:
at least 40 wt.-%, preferably 40 to 70 wt.-%, of at least one C1-C10 alkyl methacrylate, preferably methyl methacrylate;
5 to 45 wt.-%, preferably 20 to 45 wt.-%, preferably 25 to 42 wt.-%, of at least one C1-C10 alkyl acrylate, preferably butyl acrylate;
0 to 2 wt.-%, preferably 0.1 to 2 wt.-%, more preferably 0.5 to 1 wt.-%, of at least one crosslinking monomer; and
0 to 15 wt.-%, preferably 0 to 10 wt.-%, more preferably 0.5 to 5 wt.-%, of optionally further monomers.

In a more preferred embodiment, the impact modifier B is selected from particulate multiphase graft copolymers, preferably having a core-shell structure, comprising (preferably consisting of):
BC 10 to 95% by weight, based on the total graft copolymer, of a soft elastomeric core BC, having a glass transition temperature T_{g} below −10° C., which is built up from:
   BC.1 50 to 99.5% by weight, based on BC, of at least one C1-C10 alkyl acrylate, preferably n-butyl acrylate;
   BC.2 0.5 to 5% by weight, based on BC, of at least one crosslinking monomer, having two or more ethylenically unsaturated groups; and
   BC.3 0 to 10% by weight, based on BC, of at least one further ethylenically unsaturated, free radically polymerizable monomer; and
BS 5 to 90% by weight, based on the total graft copolymer, of a hard shell BS, having a glass transition temperature T_{g} above 70° C., which is built up from:
   BS.1 80 to 100% by weight, based on BS, of at least one C1-C6 alkyl methacrylate, preferably methyl methacrylate, and
   BS.2 0 to 20% by weight, based on BS, of at least one further ethylenically unsaturated, free radically polymerizable monomer, e.g. selected from C1-C6 alkyl acrylate, such as methyl acrylate, ethyl acrylate, or butyl acrylate.

For example, the crosslinking monomer BC.2 may be selected from bifunctional (meth)acrylates, tri- or multifunctional (meth)acrylates, and other known crosslinkers, such as allyl methacrylate, allyl acrylate, and divinylbenzenes. Suitable cross-linking monomers are for example describes in WO 02/20634 and EP 0 522 351.

For example, bifunctional (meth)acrylates are di-esters of (meth)acrylic acid and a poly-functional alcohol, e.g. di(meth)acrylate of propane diol, butane diol, hexane diol, octane diol, nonane diol, decane diol, eicosane diol, ethylene glycol, diethylene glycol, triethylene glycol, tetraethylene glycol, dodecaethylene glycol, tetradecaethylene glycol, propylene glycol, dipropyl glycol, tetradecapropylene glycol. For example, tri- or multi-functional (meth)acrylates are tri- or multi-esters of (meth)acrylic acid and a poly-functional alcohol, e.g. trimethylolpropane tri(meth)acrylates and pentaerythritol tetra (meth)acrylate.

For example, cyanurate und isocyanurate derivatives, such as triallyl cyanurate, triallyl isocyanurate, and crosslinking compounds as described in EP 2 380 928, can be used as crosslinking monomer.

Preferably, the crosslinking monomer BC.2 is selected from ethylene glycol methacrylate, 1,4-butanediol dimethacrylate, divinylbenzene, and allyl(meth)acrylate. More preferably the crosslinking monomer is allyl methacrylate.

Typically, the particulate multiphase graft copolymer may be formed by crosslinked particles having core-shell structure as described above, wherein the particles have an average particle diameter, determined by photon correlation spectroscopy according to DIN ISO 13321:199, in the range of 20 nm to 500 nm, preferably 50 nm to 450 nm, more preferably 100 nm to 400 nm and most preferably between 100 nm to 200 nm.

Polymeric Component C

In a preferred embodiment the acrylic-based polymer material comprises 0.0 to 50.0 wt.-%, preferably 1.0 to 40.0 wt.-%, more preferably 5.0 to 35.0 wt.-%, also preferably 10.0 to 30.0 wt.-%, also preferably from 10.0 to 50.0 wt.-%, based on the total polymer material, of one or more polymeric components C, which are different from A and optionally B and D. In particular the optional polymeric component C is compatible, i.e. homogenous miscible, with the alkyl(meth)acrylate polymer A and may form a homogenous polymer matrix together with the alkyl(meth)acrylate polymer A. and optional the components D and/or E. More preferably, said polymer blend (respectively polymer matrix) is transparent in the visible range of electromagnetic spectrum.

In particular, the optional polymeric component C is selected, so that it increases or does not significantly lower the UV transmittance of the acrylic-based polymer material in the desired range, e.g. from 260 to 300 nm, preferably from 260 to 280 nm, more particularly at 265 nm.

Preferably, the optional polymeric component C is selected from polyvinylidene fluoride (PVDF) and/or higher polyethylene glycols (PEG). e.g. polyethylene glycols having a molecular weight of at least 10,000 g/mol, preferably in the range of 10,000 to 200,000 g/mol.

Polyvinylidene fluorides (PVDF) are commonly known, often semi-crystalline, thermoplastic fluoroplastics, which are obtained via polymerization of vinylidene fluoride CH₂═CF₂ (1,1-difluoro ethylene), optionally together with suitable comonomers. Typically, PVDF may be transparent in thin layers and appears milky white at higher thickness. Generally, PVDF is synthesized by the free radical polymerization in suspension or emulsion under controlled conditions of pressure and of temperature, e.g. at a temperature from 10-150° C. and pressure of 10-300 atm. For example, PVPF is often used for the production of films or sheets.

Preferably, the PVDF, used as polymeric component C, may be selected from vinylidene fluoride homopolymers, as well as copolymers or terpolymers of vinylidene fluoride, wherein typically the amount of vinylidene fluoride units is at least 50 wt.-%, preferably at least 60 wt.-%, more preferably at least 70 wt.-%, based on the total weight of all monomer units in the PVDF. For example co- and/or terpolymers of vinylidene fluoride may be obtained by polymerization of vinylidene fluoride together with one or more comonomers, selected from partly or fully fluorinated olefins, such as vinyl fluoride, trifluoro ethylene, tetrafluoro ethylene, 3,3,3-trifluoro-1-propylene, 1,2,3,3,3-pentafluoropropylene, 3,3,3,4,4-pentafluoro-1-butylene, hexafluoro propylene, and hexafluoro isobutylene; partly or fully chlorinated fluoro-olefins, such as chlorotrifluoroethylene; perfluorinated vinyl ethers, such as perfluoro methyl vinyl ether, perfluoro ethyl vinyl ether, perfluoro-n-propyl vinyl ether, and perfluoro-2-propoxypropyl vinyl ether; other fluorine-containing monomers, such as fluorovinyl sulfonic acid; olefins, such as ethylene or propylene.

According to a preferred embodiment the polymeric component C comprises (preferably consists of) PVDF, wherein the PVDF is selected from homopolymers of vinylidene fluoride, and copolymers of vinylidene fluoride with one or more comonomer selected from vinyl fluoride, trifluoro ethylene, tetrafluoro ethylene, hexafluoro propylene, hexafluoro isobutylene, chlorotrifluoro ethylene, perfluoro methyl vinyl ether, and fluorovinyl sulfonic acid; more preferably selected from tetrafluoro ethylene, hexafluoro propylene, and chlorotrifluoro ethylene.

The mass average molecular weight of the PVDF used as polymeric component C is preferably 50.000 to 450,000; more preferably 100,000 to 400,000; and even more preferably 110,000 to 300,000. Typically, the mass average molecular weight of the PVDF can be measured via gel permeation chromatography (GPC), using dimethyl formamide as a solvent and calibration using polystyrene standard.

Typically, the alkyl(meth)acrylate polymer A and PVDF can be melt blended to form a homogeneous blend, using appropriate mixing ratios. For example, PVDF as described in EP 2046888. US 2016/0200884 A1 and WO 2009/000566 can be used in the present invention.

Typically, for the purposes of the invention commercially available grades of PVDF may be utilized, such as Kureha KF polymers from Kureha Corporation, Japan (e.g. KF TH850, KF TH1000, and KF TH1100), Kynar® grades from Arkema (e.g. Kynar® 760, Kynar® 740, Kynar® 720, and Kynar® 710), 3M Dyneon® grades from Dyneon, and Solef® grades from Solvay (e.g. Solef®1006, 1008, 1015, 5140, 6008, 6010, 6012, 60512, 11008, 21508, 11010.21510).

According to another preferred embodiment the polymeric component C comprises (preferably consists of) higher polyethylene glycols (PEG). Polyethylene glycols (PEG) are commonly known hydrophilic, biocompatible polymers, which are often used in medical application. Generally, PEG is prepared by a ring-opening polymerization of ethylene oxide. Typically, PEGs are available in a broad range of molecular weights and molecular weight distribution (from polydisperse to discrete PEGs). As a rule, low molecular weight PEG, e.g. PEG having a molecular weight less than 10,000 g/mol, are commonly known plasticizers, e.g. utilized as component D. Preferably, polyethylene glycol (also referred to as polyethylene oxide) used as polymeric component C has a molecular weight of at least 10,000 g/mol, preferably from 10,000 to 200,000 g/mol, more preferably from 10,000 to 100.000 g/mol. Typically, the molecular weight of PEGs refers to the weight averaged molecular weight.

Typically, for the purposes of the invention commonly known, commercially available grades of PEG may be utilized, for example PEG 10,000 or PEG 20,000, wherein the number indicates the molecular weight, typically given as weight average molecular weight.

Typically, the alkyl(meth)acrylate polymer A and PEG, as well as the alkyl(meth)acrylate polymer A, PEG, and PVDF, can be melt blended to form a homogeneous blend, using appropriate mixing ratios.

In a particular preferred embodiment, the optional polymeric component C is used to adjust the refraction index of the polymer matrix (i.e. formed by components A, C and optional D) in view of the refraction index of the impact modifier B, preferably the particulate multiphase graft copolymer based on an elastomeric crosslinked alkyl(meth)acrylate core (e.g. a crosslinked butyl acrylate as described above). For example, the weight ratio of components A:C can be in the range of 1:1 to 5:1, preferably 3:1 to 4:1.

According to a preferred embodiment, the alkyl(meth)acrylate polymer A comprises, based on its total weight, at least 50.0 wt.-% methyl methacrylate; the polymeric component C is selected from polyvinylidene difluoride (PVDF), and the ratio of the components A:C is in the range of 3:1 to 4:1, preferably of 3.3:1 to 3.5:1, more preferably about 3.4:1.

According to a preferred embodiment, the acrylic-based polymer material comprises:
at least 50 wt.-%, preferably at least 60 wt.-%, more preferably at least 70 wt.-%, based on the acrylic-based polymer material, of at least one alkyl (meth)acrylate polymer A,
at least one impact-modifier B, selected from particulate multiphase graft copolymers, which comprises an elastomeric phase built up from crosslinked C1-C10 alkyl acrylate, preferably n-butyl acrylate, and a hard, outer shell comprising at least one C1-C10 alkyl (meth)acrylate, preferably methyl methacrylate; and at least one polymeric component C selected from polyvinylidene difluoride (PVDF),
wherein the ratio of the components A:C is in the range of 3:1 to 4:1, preferably of 3.3:1 to 3.5:1, more preferably about 3.4:1.

Preferably, the acrylic-based polymer material comprises (preferably consists of), based on the total weight of the acrylic-based polymer material:
A. 50.0 to 94.0 wt.-%, preferably 50.0 to 85.0 wt.-%, more preferably 50.0 to 75 wt.-% of at least one alkyl(meth)acrylate polymer A, preferably a polyalkyl(meth)acrylate comprising 80.0 to 100.0 wt.-%, preferably 90.0 to 100.0 wt.-%, more preferably 95.0 to 100.0 wt.-% methyl methacrylate (MMA), and 0.0 to 20.0 wt.-%, preferably 0.0 to 10.0 wt.-%, more preferably 0.0 to 5.0 wt.-% of an alkyl(meth)acrylate other than MMA, each based on the weight of the polyalkyl(meth)acrylate;
B. 5.0 to 35.0 wt.-%, preferably 10.0 to 30.0 wt.-%, more preferably 15.0 to 30 wt.-%, of at least one impact modifier B, preferably selected from a particulate multiphase graft copolymer, which comprises a elastomeric phase built up from crosslinked C1-C10 alkyl acrylate, preferably n-butyl acrylate, and a hard, outer shell comprising at least one C1-C10 alkyl (meth)acrylate, preferably methyl methacrylate;
C. 1.0 to 30.0 wt.-%, preferably 5.0 to 25.0 wt.-%, more preferably 10.0 to 20.0 wt.-%, of at least one polymeric component C, different from A and B, preferably selected from polyvinylidene fluoride (PVDF);
D. 0.0 to 20.0 wt.-%, preferably 0.0 to 10.0 wt.-%, of at least one plasticizer D, and
E. 0.0 to 10.0 wt.-%, preferably 0.0 to 5.0 wt.-%, of at least one further component E, preferably selected from additives, auxiliaries and/or fillers.

According to another preferred embodiment, the alkyl(meth)acrylate polymer A comprises, based on its total weight, at least 50.0 wt.-% methyl methacrylate; the polymeric component C is selected from higher polyethylene glycols (PEG), e.g. polyethylene glycols having a molecular weight in the range of 10,000 to 200,000 g/mol, and the ratio of the components A:C is in the range of 0.8:1 to 5:1, preferably of 1:1 to 3:1, more preferably about 1:1 to 2:1.

According to another preferred embodiment, the alkyl(meth)acrylate polymer A comprises, based on its total weight, at least 50.0 wt.-% methyl methacrylate; the polymeric component C is a combination of at least one PVDF as described above, and at least one PEG, as described above.

For example, the acrylic-based polymer material comprises (preferably consists of) a polymeric matrix, which is formed by a homogenous blend comprising (preferably consisting of) the components A and C as described above, wherein said polymeric matrix comprises, each based on the total amount of the polymeric matrix:
50 to 100 wt.-%, preferably 50 to 89 wt.-%, more preferably 50 to 80 wt.-%, of the alkyl(meth)acrylate polymer A;
0 to 40 wt.-%, preferably 10 to 35 wt.-%, more preferably 15 to 30 wt.-%, of at least one PVDF;
0 to 50 wt.-%, preferably 1 to 40 wt.-%, more preferably 5 to 35 wt.-%, of at least one PEG.

Plasticizer D

In a preferred embodiment the acrylic-based polymer material comprises 0.0 to 20.0 wt.-%, preferably 0.0 to 10.0 wt.-%, more preferably 0.0 to 5.0 wt.-%, also preferably 0.1 to 20.0 wt.-%, also preferably from 1.0 to 10.0 wt.-%, based on the total polymer material, of one or more plasticizers D.

Typically, ductility, craze resistance and/or chemical resistance of the acrylic-based polymer material and of the formed article made thereof can be improved by the addition of one or more appropriate plasticizers as component D. Plasticizers per se are familiar to the skilled person and described for example in Ullmann's Encyclopaedia of Industrial Chemistry, 2012, Plasticisers, D. F. Cadogan etc. For the purpose of the present invention, the plasticizers D usually have a molecular weight of less than 10,000 g/mol, and a melting temperature of not more than 40° C.

If a polymeric compound is used as a plasticizer in the acrylic-based polymer material, such polymeric compound should ideally have a glass transition temperature T_{g} of not more than 40° C., as measured according to standard ISO 11357-2:2013 and/or an weight averaged molecular weight of not more than 200,000 g/mol, preferably less than 10,000 g/mol.

Furthermore, to ensure that presence of the plasticizers does not adversely affect optical properties of the acrylic-based polymer material, the plasticizer should be miscible with the acrylic-based polymer material.

Examples of particularly suitable plasticizers include in particular polyethylene glycol having a molecular weight from 500 to <10,000 g/mol, tributyl citrate, butyl lactate, and 1,2-cyclohexane dicarboxylic acid diisononyl ester (commercially available as a mixture of isomers under the trademark name Hexamoll® DINCH from BASF SE, Ludwigshafen, Germany). 1,2-cyclohexane dicarboxylic acid diisononyl ester is typically a mixture of isomers and usually comprises 10 wt.-% n-nonylalcohol, 35 to 40 wt.-% methyloctylalcohol, 40 to 45 wt.-% dimethylheptylalcohol und 5 to 10 wt.-% methylethylhexylalkohol, based on the total weight of isononyl alcohol residues.

In a preferred embodiment, the acrylic-based polymer material comprises 0.01 to 20.0 wt.-%, preferably from 0.1 to 15.0 wt.-%, more preferably from 1.0 to 10.0 wt.-%, based on the total polymer material, of at least one polyethylene glycol having a molecular weight from 500 to <10,000 g/mol, as plasticizer D.

Further Component E

The acrylic-based polymer material utilized in the inventive method for UV sterilization may comprise an optional further component E, which is different from components B, C and D. Preferably, the further component E is selected from non-polymeric components. Typically, the further component E is selected from common additives, such as flow improvers, stabilizers, mold release agents etc. in usual amounts, in order to adjust mechanical and/or optical properties, as long as the transmittance of the polymeric materials in the relevant range of wavelength, i.e. 260 nm to 300 nm, preferably at 265 nm, is not less than 10%.

Typically, the further component E may be present in an amount of 0.0 to 15.0 wt.-%, preferably 0.0 to 10.0 wt.-%, more preferably 0 to 5 wt.-%. Preferably, the further component E may be present in an amount of 0.0001 to 5.0 wt.-%, also preferably 0.001 to 2.0 wt.-%, based on the total polymer material.

Preferably, the acrylic-based polymer material may comprise as optional further component E, one or more additive selected from external lubricants, antioxidants, flame retardants, further hindered amine light stabilizers (HALS), flow improvers such as stearyl alcohol or palmitic acid, metal additives for screening against electromagnetic radiation, antistatic agents, mold release agents, dyes, pigments, adhesion promoters, anti-weathering agents, heat stabilizers, UV stabilizers, UV absorbers, gamma ray stabilizers, antioxidants, and fillers, provided that the UV transmittance of the acrylic-based polymer material in the relevant range is not adversely affected by these additives.

Lubricants and mold release agents which can reduce or completely prevent possible adhesion of the molding material to the injection mold are important for the injection molding process and may preferably be employed.

For example, lubricants, selected from the group consisting of saturated fatty acids having less than 20, preferably 16 to 18, carbon atoms or of the saturated fatty alcohols having less than 20, preferably 16 to 18, carbon atoms, may be present as auxiliaries. For example, stearic acid, stearyl alcohol, palmitic acid, palmitic alcohol, lauric acid, lactic acid, glycerol monostearate, pentaerythrol, and industrial mixtures of stearic and palmitic acid. Also suitable are n-hexadecanol, n-octadecanol and industrial mixtures of n-hexadecanol and n-octadecanol. A particularly preferred lubricant or mold release agent is stearyl alcohol.

The lubricants are typically used in amounts of not more than 0.35 wt.-%, for example 0.05 wt.-% to 0.25 wt.-% based on the weight of the acrylic-based polymer material.

In a preferred embodiment, at least one sterically hindered amine may be used as component E, giving an improvement in resistance to weathering, yellowing or degradation of the polymer material. Especially preferred sterically hindered amines include dimethylsuccinate-1-(2-hydroxyethyl)-4-hydroxy-2,2,6,6-tetramethylpiperazine polycondensate, poly[{6-(1,1,3,3-tetramethylbutyl)amino-1,3,5-triazine-2,4-diyl}{(2,2,6,6-tetramethyl-4-piperidyl)imino}-hexamethylene{(2,2,6,6-tetramethyl-4-piperidyl)imino}], N,N′-bis(3-aminopropyl)ethylene-diamine-2,4-bis[N-butyl-N-(1,2,2,6,6-pentamethyl-4-piperidyl)amino]-6-chloro-1,3,5-triazine condensate, bis(2,2,6,6-tetramethyl-4-piperidyl)sebacate and 2-(3,5-di-t-4-hydroxybenzyl)-2-n-butylmalonate bis(1,2,2,6,6-pentamethyl-4-piperidyl).

In a preferred embodiment, the acrylic-based polymer material is free of any UV absorber and/or UV stabilizer. Further, it is preferred, that the amount of UV absorber and/or UV stabilizer is less than 100 ppm, preferably less than 10 ppm, based on the acrylic-based polymer material.

However, it is possible that the acrylic-based polymer material comprises a small amount of specific UV absorbers, that do not significantly reduce the transmittance at the desired UV wavelength range, e.g. from 260 to 300 nm, preferably from 260 to 280 nm, more particularly at 265 nm. Typically, the acrylic-based polymer material may comprise up to 0.3 wt.-%, preferably up to 0.1 wt.-%, for example 0.0001 to 0.1 wt.-%, preferably from 0.005 to 0.05 wt.-%, based on the weight of the acrylic-based polymer material of specific UV absorbers as component E.

Particular preferred UV absorbers may be, for example, derivatives of benzophenone (e.g. 2-hydroxy-4-n-octyloxybenzophenone, 2,4-dihydroxybenzophenone, 2,2′-dihydroxy-4-methoxybenzophenone, 2,2′,4,4′-tetrahydroxybenzophenone, 2,2′-dihydroxy-4,4′-dimethoxybenzophenone, 2-hydroxy-4-methoxybenzophenone), benzotriazoles (e.g. 2-(2′-hydroxy-5′-methyl-phenyl)benzotriazole, commercially available as Tinuvin® P, from BASF SE; Ludwigshafen, Germany, or 2-(2′-hydroxy-3′-dodecyl-5′-methy-decyl)benzotriazole, substituted benzotriazoles (z.B. 2-(2-hydroxy-5-methylphenyl)benzotriazole, 2-[2-hydroxy-3,5-di(alpha,alpha-dimethylbenzyl)phenyl]benzotriazole, 2-(2-hydroxy-3,5-di-tert.-butylphenyl)benzotriazole, 2-(2-hydroxy-3,5-di-tert.-amylphenyl)benzotriazole, 2-(2-hydroxy-5-tert.-butylphenyl)benzotriazole, 2-(2-hydroxy-3-sec-butyl-5-tert.-butylphenyl)benzotriazole and 2-(2-hydroxy-5-tert.-octylphenyl)benzotriazole).

2-(2′-hydroxy-5′-methyl-phenyl)benzotrazole (commercially available as Tinuvin® P), is particularly preferred due its low absorbance in the UV-C range. In a preferred embodiment, the acrylic-based polymer material comprises 0 to 0.3 wt.-%, preferably 0 to 0.1 wt.-%, also preferably from 0.0001 to 0.1 wt.-%, more preferably from 0.005 to 0.05 wt.-%, based on the weight of the acrylic-based polymer material, of at least one benzotriazole type UV absorber, more preferably 2-(2′-hydroxy-5′-methyl-phenyl)benzotriazole (Tinuvin® P) as component E.

Other suitable UV absorbers may be, for example, oxanilide and commonly known derivatives thereof, for example N-(2-ethoxyphenyl)-N′-(2-ethylphenyl) ethane diamide, which is commercially available from BASF SE as Tinuvin® 312.

Another suitable UV absorber is selected from known sterically hindered phenolic compounds, for example octadecyl-3-(3,5-di-tert-butyl-4-hydroxyphenyl)propionate, which is commercially available from BASF SE under the product name IRGANOX 1076.

As will be readily appreciated by a skilled person, mixtures of chemically different UV absorbers may also be employed.

Examples of suitable free radical scavengers/UV stabilizers include inter alia sterically hindered amines, which are known by the name HALS ((Hindered Amine Light Stabiliser) (for example described in Kunststoffe [Plastics], 74 (1984) 10, pages 620 to 623; Farbe+Lack [Paints+Finishes], 96^{th}, year, 9/1990, pages 689 to 693). Often, such sterically hindered amines do not absorb in the UV range. They trap free radicals formed, which once again the UV absorbers are incapable of doing. Examples of suitable HALS stabilizers are bis(2,2,6,6-tetramethyl-4-piperidyl) sebacate, 8-acetyl-3-dodecyl-7,7,9,9-tetramethyl-1,3-8-triazaspiro(4,5)decane-2,5-dione, bis(2,2,6,6-tetramethyl-4-piperidyl)succinate, poly(N-β-hydroxyethyl-2,2,6,6-tetramethyl-4-hydroxypiperidine succinic acid ester) and bis(N-methyl-2,2,6,6-tetramethyl-4-piperidyl) sebacate, and mixtures thereof.

The free radical scavengers/UV stabilizers can be used in the acrylic-based polymer material in amounts of 0.01 to 1.5 wt.-%, especially in amounts of 0.02 to 1.0 wt.-%, in particular in amounts of 0.02 to 0.5 wt.-%, based on the total polymer material.

Properties of the Acrylic-Based Polymer Material

As already mentioned, the acrylic-based polymer material or the formed article made thereof can advantageously be sterilized, including sterilization of the inner surface, using UV radiation having a wavelength in the range of 260 nm to 300 nm, more preferably using UV radiation having a wavelength of 265 nm.

The transmittance of the acrylic-based polymer material at the wavelength of 265 nm or in the range of 260 to 300 nm is measured using an instrument as defined in the standard ISO 13468-2 which is suitable for measurements at this wavelength (e.g. a Varian Cary 5000 spectrophotometer). For the measurement a test plaque of the following dimensions can be used: 2×3 inches (5.08×7.62 cm), thickness of 0.125 inch (3.175 mm).

Additionally, the acrylic-based polymer material or the formed article made thereof has an excellent transparency and a substantially non-cloudy appealing appearance. In particular, haze of the acrylic-based polymer material measured at 23° C. on an injection molded specimen having a thickness of 3 mm according to standard ASTM D1003 (2013) is lower than 50%, preferably lower than 40%, more preferably lower than 30% even more preferably lower than 20%.

Furthermore, the acrylic-based polymer material preferably shows a light transmittance, TD65 according to DIN 5033-7 (2014) of at least 80%, preferably at least 90%, measured at 23° C. on an injection molded specimen having a thickness of 3 mm.

The Vicat softening temperature of the acrylic-based polymer material according to ASTM D-1525, (Method B, 5.0 kG 60° C./hr or FT/DS Correlation) is advantageously at least 80° C., preferably at least 90° C., more preferably at least 100° C.

The nominal elongation at break of the acrylic-based polymer material according to ASTM D-638 should preferably be at least 3.0%, particularly preferably at least 4.0%.

Due to its advantageous rheological properties, the acrylic-based polymer material as described is highly suitable for manufacturing of medical grade articles by means of injection molding or other suitable thermoforming process. The acrylic-based polymer material typically has a melt volume flow rate MVR measured according to ASTM D-1238 at 230° C. and 3.8 kg, of greater than 0.5 cm³/10 min, preferably greater than 1.0 cm³/10 min, more preferably greater than 1.2 cm³/10 min, most preferably in the range from 1.0 to 10.0 cm³/10 min.

UV Exposure (Step b)

The inventive method for UV sterilization encompasses the exposure of the outer surface of the formed article with UV radiation at a wavelength in the range of 260 to 300 nm, preferably with UV radiation at a wavelength of 265 nm, wherein at least one part of said formed article is made from (meaning consists of) an acrylic-based polymer material as described, and wherein the UV radiation is typically provided by one or more appropriate UV radiation source, preferably selected from UV LEDs.

Typically, the exposure with UV radiation is carried out in any suitable equipment that allows an appropriate arrangement of the UV radiation source and the formed article that is sterilized. If necessary, such equipment may include a suitable housing made from a non-UV transmissive material in order to avoid undesirable UV exposure to the environment.

Particularly, exposure of the formed article with UV radiation in the range of 260 to 300 nm means that the at least one part of the formed article, which is made of the acrylic-based polymer material, is exposed to said UV radiation.

In a preferred embodiment the whole formed article is made from the acrylic-based polymer material as described, and the UV sterilization step b) encompasses the exposure of the whole formed article or of at least a part of the formed article with UV radiation in the range of 260 nm to 300 nm.

Typically, the inventive method may also encompass the sterilization of the outer surface of the formed article and/or of parts that are not made of the acrylic-based polymer material caused by the UV radiation. However, the main advantage of the inventive method is that effective sterilization of the inner surface of the formed article, which is not directly exposed to the UV radiation, is achieved.

Preferably, the desired reduction or inhibition of growth of microorganisms, for example as described below, is achieved by the inventive method for UV sterilization. Further, it is possible to combine the inventive method for UV sterilization with one or more commonly known, physical or chemical processes of sterilization, disinfection and/or decontamination, such as chemical sterilization and/or disinfection using commonly known disinfectants, e.g. ethylene oxide, vaporized hydrogen peroxide, ozone, alcohol/water mixtures; irradiation with gamma radiation, electron beam or X-ray; heat and/or steam treatment.

Furthermore, according to another preferred embodiment of the invention it is possible that the formed article, preferably a medical device, more preferably a disposable medical device, is surrounded by an UV-transmissive material, e.g. a polymeric protective packaging, during the UV exposure step b. In particular “UV transmissive” means that the material has a transmittance of at least 10%, averaged over the wavelength range from 260 nm to 300 nm, and measured in accordance with ISO 13468-2 at a thickness of 3 mm. In a preferred embodiment, the formed article, preferably a medical device, more preferably a disposable medical device, is surrounded by a protective packaging during the UV exposure step b, wherein the protective packaging is made of an UV-transmissive polymer material, preferably selected from polyethylene PE, polypropylene PP, polyvinylchloride PVC (in particular PVC without phthalate plasticizer, such as di(2-ethylhexly)phthalate DEHP, referred to as non-DEHP-PVC), polysiloxane (silicone), and acrylic-based polymer material as described in the present invention. Preferably, such protective packaging is impervious to microorganisms.

It is also possible that the formed article is placed in a container made of UV transmissive quartz glass during UV exposure. Further, the formed article, preferably a medical device, more preferably a medical device surrounded by a protective packaging, is placed in a second outer packaging/container after UV exposure.

UV Radiation Source

The choice of the ultraviolet (UV) radiation source for use in the inventive method is not particularly limited, as long as the UV radiation source emits UV radiation at a wavelength in the range of 260 nm to 300 nm, preferably 260 to 280 nm. It is preferred but not necessary that at least a significant amount of emission spectrum of the UV source, preferably selected from UV light emitting diodes (UV-LEDs), is in the range of 260 nm to 300 nm, preferably 260 to 280 nm. More preferably, the UV source has at least one emission peak falling within said range.

The emission peak half width of the UV radiation source is typically not higher than 30 nm, preferably not higher than 20 nm, more preferably not higher than 15 nm. Preferably, the operating temperature of the UV radiation source is compatible with the employed polymeric material. Ideally, the UV radiation source is substantially monochromatic i.e. has a single emission peak. The examples of suitable UV radiation source include a UV light emitting diode (UV-LED), an excimer laser, a plasma or synchrotron source or a gas discharge tube.

Typically, UV mercury lamps only emit light at 254 nm, so it is preferred to use UV light-emitting diodes (UV-LEDs) which can be configured to emit certain target wavelengths, i.e. desired UV wavelength from 260 to 300 nm, preferably from 260 to 280 nm, more particularly at 265 nm, in the inventive method. Further, UV mercury lamps have additional disadvantages, such as a long warm-up time, and risk of mercury exposure. Thus, the use of UV-LEDs is particularly preferred. In a particularly preferred embodiment, one or more UV-C LEDs are used as UV radiation source in the inventive method for UV sterilization. It is advantageous to use UV-C LEDs as it allows adjusting and combination of the most effective wavelengths in view of the acrylic-based polymer material as well as target microorganisms. Generally, each microorganism requires a specific UV dosage depending on the UV wavelength and based on the desired log reduction.

In some embodiments of the present invention, the UV radiation source is a pulsed UV radiation source, e.g. a pulsed UV-LED.

Particularly, the UV radiation source is selected from commonly known and commercially available germicidal UV light sources, e.g. germicidal UV-LEDs, showing emission, particularly an emission peak, in the desired wavelength range, e.g. from 260 to 300 nm, preferably from 260 to 280 nm, more particularly at 265 nm. Ideally, the emission spectrum of the UV radiation source comprises an emission peak or a single emission peak in the range of 260 nm to 300 nm, preferably at 265 nm. For example, these LEDs are commercially available as Klaran® UV-C LEDs from Crystal IS Inc. (USA); as UV-C-LED having a wavelength of 265 nm from Stanley Electric Co.; or as Oslon UV 3636 from Osram Opto Semiconductors GmbH.

Preferably, one or more UV LEDs, selected from UV LEDs emitting at 266 nm, 270 nm, 275 nm, 279 nm, 280 nm, or 285 nm, were utilized in the inventive method. More preferably at least one UV LED emitting at 265 nm were utilized in the inventive method.

Generally, the UV irradiance or also referred to as UV intensity (given in W/m² or mW/cm²) refers to the irradiance field of an UV irradiation system including the UV radiation source and the surface exposed to said UV radiation, i.e. means the total radiant flux (energy) incident on a surface from all directions. The UV irradiance greatly depends on the distance from the UV source and the transmittance of the medium. Preferably, the UV irradiance in the inventive method for sterilization provided by the UV radiation source, preferably by one or more UV LEDs, is in the range of 0.1 to 1.0 mW/cm², preferably 0.2 to 0.5 mW/cm² (relating to the outer surface of the formed article).

Generally, the UV dose (given in mJ/cm² or J/m²) refers to the amount of UV irradiation incident on a surface or in particular absorbed by an exposed population of microorganisms. The UV dose results from the UV irradiance of the UV radiation source and the exposure time (in seconds). Generally, the required UV dose depends on the UV wavelength range used, the target microorganism(s) and the required reduction of growth or number of microorganisms, which is typically given in CFU log reductions.

Preferably, the exposure of the outer surface of the formed article with UV radiation is carried out in such way that a UV dose in the range of 1 to 10,000 mJ/cm², preferably 2 to 2,000 mJ/cm², also preferably 10 to 1,000 mJ/cm² is effected (relating to the outer surface of the formed article). It was found that such UV dose is often suitable to effect the desired reduction of microorganism at the inner surface of a formed article, wherein the formed article has a thickness of less than 3.0 mm, preferably less than 1.0 mm, more preferably less than 0.5 mm, for example in the range from 0.01 to 4 mm, preferably in the range from 0.1 to 1 mm.

Typically, in the inventive method for UV sterilization the distance between the UV radiation source and the outer surface of the formed article is adjusted so that the desired sterilization effect, i.e. the desired CFU log reduction, is obtained.

Typically, the UV radiation exposure is carried out one or several times, each cycle is about 10 to 600 seconds, preferably 60 to 600 seconds. Typically, the exposure time is selected depending on the desired sterilization effect, i.e. the desired CFU log reduction, and/or the UV radiation source.

Reduction of Microorganisms Generally, sterilization results in colony forming units (CFU) log reduction ≥6 (i.e. reduction of ≥10⁶ CFU) of present microorganisms, including the most resistant spores. Generally, disinfection results in CFU log reduction ≥3 of present microorganisms, not including spores. Generally, decontamination achieves minimum log reduction of ≥1 CFU of microorganisms. In terms of the present invention the term “sterilization” or “method for sterilization” includes sterilization, disinfection and/or decontamination.

According to a preferred embodiment, the number of colony forming units (CFU) of one or more relevant microorganisms, for example mentioned below, is reduced/inhibited by at least 1 log, preferably at least 2 log, preferably at least 3 log, preferably at least 4 log, also preferably at least 5 log, even more preferably of at least 6 log, using the inventive method for UV sterilization. As a skilled person known, the desired log reduction can for example be obtained by adjusting exposure time and/or UV dose.

Generally, the term microorganisms include bacteria, fungi, algae, viruses, archaea, protozoa. Preferably, the inventive method for UV sterilization is directed to reduction of microorganisms selected from bacteria, fungi (in particular yeast), and viruses, more preferably selected from bacteria and/or yeast.

Typically, the inventive method for UV sterilization can inhibit the growth of one or more microorganisms, which is relevant for the specific intended use of the formed article, e.g. use as medical device or as food container. For example, the inventive method for UV sterilization may inhibit the growth of one or more microorganisms, for example of genus selected from Escherichia, Salmonella, Listera, Aspergillus, Bacillus, Cryptosporidium, Clostridium, Streptomyces, Aeromonas, Candida, Helicobacter, Klebsiella, Legionella, Listeria, Pseudomonas, Staphylococcus, Streptococcus, Lactobacillus, Bifidobacterium, Oenococcus, and Saccharomycodes.

For example, the inventive method for UV sterilization may inhibit the growth of one or more species of microorganisms, selected from Escherichia coli, Salmonella typhimurium, Listeria monocytogenes, Cryptosporidium, Aspergillus niger, Bacillus anthracis, Bacillus cereus, Bacillus megaterium, Bacillus subtilis, Clostridium pasteurianum Streptomyces griseus, Aeromonas hydrophila, Candida aurs, candida, Helicobacter pylon, Klebsiella pneumoniae, Legionella pneumophila, Listena monocytogenes, Pseudomonas aeruginosa, Staphylococcus aureus, Streptococcus hemolyticus, Oenococcus oeni, Lactobacillus acidophilus, Lactobacillus plantarum, Bifidobacterium lactis, Bifidobac bifdum, Lactobacillus rhamnosus, and Bifidobacterium breve.

Typically, in the inventive method for UV sterilization a CFU log reduction of up to 4 can be obtained for microorganism Escherichia coli using an UV LED having emission peak at 265 nm utilizing a UV dose in the range of 3 to 20 mJ/cm², wherein the CFU log reduction is effected at the inner surface of the formed article.

Formed Article

In another aspect, the present invention is directed to a formed article for use in the inventive method for UV sterilization, preferably selected from medical devices; containers and/or packages used for cosmetic or pharmaceutical products or used in food industry, wherein at least one part of said formed article is made from an acrylic-based polymer material, which comprises at least 50 wt.-%, preferably at least 60 wt.-%, more preferably at least 70 wt.-%, based on the acrylic-based polymer material, of at least one alkyl(meth)acrylate polymer A; and wherein the acrylic-based polymer material has a transmittance of at least 10%, preferably at least 15%, more preferably at least 20%, also preferably at least 25%, averaged over the wavelength range from 260 nm to 300 nm, and measured in accordance with ISO 13468-2 at a thickness of 3 mm. Preferably, the inventive formed article comprises at least one inner surface, that includes openings, cavities and/or pores, which cannot or only with high difficulties be exposed to UV radiation directly.

Said formed articles can advantageously be sterilized by the inventive method for UV sterilization as described above. The description and the preferred embodiments of the inventive method for UV sterilization apply to the inventive formed article accordingly.

Preferably, the formed article or the at least one part of the formed article made of the acrylic-based polymer material has a wall thickness of less than 3.0 mm, preferably less than 1.0 mm, more preferably less than 0.5 mm, for example in the range from 0.01 to 4 mm, preferably in the range from 0.1 to 1 mm. It was found that UV transmittance through the acrylic-based polymer material as described is high enough in order to effectively reduce the growth of microorganisms at the inner surface of the formed article.

In a preferred embodiment, the formed article essentially consists of or is made of the acrylic-based polymer material as described above. For example, the formed article is made from the acrylic-based polymer material via a thermal molding process, preferably via injection molding, blow molding or thermoforming, more preferably injection molding. It is possible to effectively sterilize an injection molded article having a complex shape and/or including openings and/or cavities.

Furthermore, it is possible that the formed article comprises, besides the acrylic-based polymer material, at least one other UV-C transparent polymer material, for example selected from polyolefins, such as polyethylene, polypropylene, polyvinylchloride, such as non-DEHP-PVC. Further, it is possible that the formed article is combined (e.g. linked via screwing or adhesion) with another part made of UV-C transparent polymers, e.g. phthalate free PVC (non-DEHP-PVC) or silicone. Advantageously, such combination can be used in the inventive process for sterilization, i.e. be sterilized in one step.

Further, it is possible that the formed article comprises one or more parts or sections of any arbitrary non UV-C transparent material, as long as said non UV-C transparent parts do not prevent inner surfaces from being sterilized. For example, such non UV-C transparent part may be located at one site of the formed article, that is opposite to the part made of the acrylic-based polymer material and opposite to the UV-C radiation source.

In a particularly preferred embodiment, the formed article is a medical device, preferably a disposable medical device. Preferably, the medical device can be sterilized by the inventive method directly after its production. For example, the formed article is a medical device selected from medical diagnostic devices, intravenous and catheter accessory, blood handling devices, chest drainage units, respiratory ventilating devices, medical filter housings, permanent device housings, tubes, connectors, fittings, and cuvettes. Said devices include but are not limited to luer locks, Y-sites, spikes, fittings, nozzles, protection caps and covers, blood plasma separators, collection and specimen vessels, and adapters, catheter accessories, chest drainage units, valve assemblies, meter housings, flow controls, filter housings, drip chambers, intravenous adapters, yankauers, rigid tubes, diagnostic cuvettes, diagnostic test packs, diagnostic rotors, optical sensor viewports, microfluidic devices, bracheotherapy needle hubs, inhalation mouthpieces and spacers.

Furthermore, it is possible to use the inventive method in any other area where formed articles made of the acrylic-based polymer material as described above are applicable and where disinfection or sterilization is required or desired, e.g. in production of food and dairy products, pharmaceutic or cosmetic products, or as well as for sanitary and bath equipment.

In another preferred embodiment, the formed article is selected from containers and/or packages used for cosmetic or pharmaceutical products or used in food industry, e.g. in production and packaging of beverages, meat products, or dairy products.

The formed article according to the invention, for example for medical use, can be prepared from the acrylic-based polymer material via commonly known processes, in particular thermal molding processes or by processing via the elastoviscous state, for example kneading, rolling, calendering, extrusion, injection molding, blow molding or thermoforming, in particular injection molding, being particularly preferred here.

The injection molding of the acrylic-based polymer material can be effected in a manner known per se at temperatures in the range of 220° C.-260° C. (melt temperature) and a mold temperature of preferably 60° C. to 90° C. The extrusion is preferably carried out at a temperature of 220° C. to 260° C.

In this context, the present invention is preferably directed to a process for producing the inventive formed article, wherein the process comprises a step, selected from injection molding, blow molding and thermoforming, of the acrylic-based polymer material.

Use of Acrylic-Based Polymer Material and Formed Article in UV Sterilization

Furthermore, the present invention is directed to the use of an acrylic-based polymer material, which comprises at least 50 wt.-%, preferably at least 60 wt.-%, more preferably at least 70 wt.-%, based on the total polymer material, of at least one alkyl(meth)acrylate polymer A, for the production of formed articles, which are subject to a UV sterilization process, wherein the acrylic-based polymer material has a transmittance of at least 10%, preferably at least 15%, more preferably at least 20%, also preferably at least 25%, averaged over the wavelength range from 260 nm to 300 nm, and measured in accordance with ISO 13468-2 at a thickness of 3 mm. Preferably, the UV sterilization process includes the exposure of the outer surface of the formed article with UV radiation at a wavelength in the range of 260 nm to 300 nm.

Finally, a further aspect of the present invention is directed to the use of the inventive formed article as described above in a method for UV sterilization, wherein UV radiation at a wavelength in the range of 260 nm to 300 nm is utilized.

The description and preferred embodiments of the inventive method for UV sterilization and of the inventive formed article apply to the inventive use accordingly.

### DESCRIPTION OF THE FIGURES

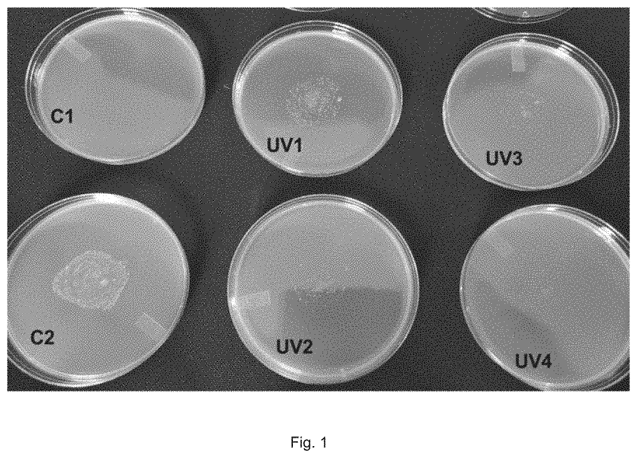

FIG. 1 illustrates the results of the UV sterilization test showing the bacterial growth on petri dished after UV exposure and incubation.

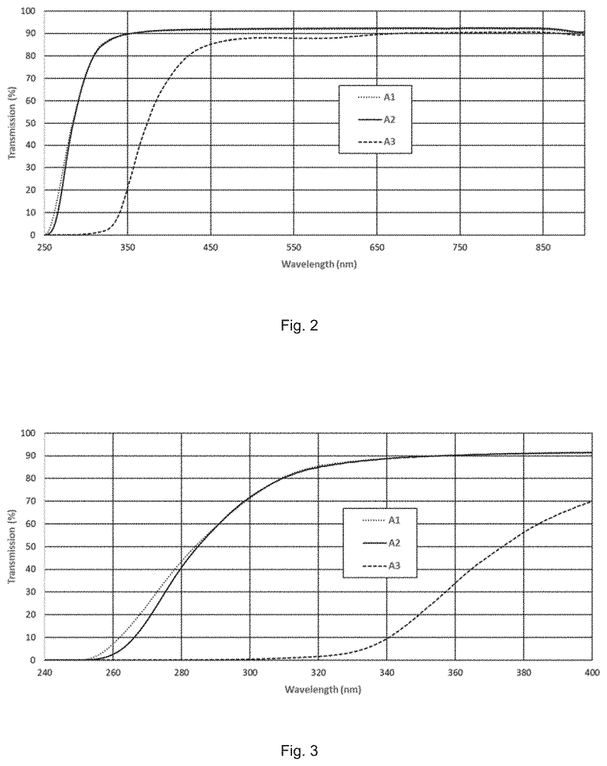

FIGS. 2 and 3 shows the transmission spectra of acrylic-based polymer materials as described in the examples. The transmission spectra show the transmission (in %) as a function of the wavelength (in nm) in the ranges of 250 to 900 nm (FIG. 2) and 240 to 400 nm (FIG. 3).

The following examples explain the present invention in detail without it being intended to limit the concept of the invention.

### Examples

I. Acrylic-Based Polymer Materials

The following acrylic based polymer materials were used:
A1: Thermoplastic alkyl(meth)acrylate polymer A1, comprising about 92 wt.-% MMA, about 8 wt.-% MA and no UV stabilizer, having a melt volume rate of about 21 cc/10 min (measured according to ASTM D-1238, at 230° C. and 3.8 kg load);
A2: Thermoplastic alkyl(meth)acrylate polymer A2, comprising about 100 wt.-% MMA, and no UV stabilizer, having a melt volume rate of about 2.2 cc/10 min (measured according to ASTM D-1238, at 230° C. and 3.8 kg load);
A3*: Thermoplastic (meth)acrylate polymer A3, impact modified copolymer of methyl methacrylate (MMA), styrene, and ethyl acrylate (EA) having a having a melt index of about 10 g/10 min (measured according to ASTM D-1238, at 230° C. and 5 kg load), wherein A2 comprises about 61 w.-% MMA. 18 wt.-% styrene, 4 wt.-% EA and 16 wt.-% of butadiene;
A4: Thermoplastic alkyl(meth)acrylate polymer A4, comprising about 96 wt.-% MMA, 4 wt.-% MA and no UV stabilizer, having a melt volume rate of 1.8-2.8 cc/10 min (measured according to ASTM D-1238, at 230° C. and 3.8 kg load).

Test specimens (plaques of 2×3 inches, thickness of 0.125 inch/3.175 mm) were prepared from the acrylic-based polymer materials A1 to A4 via injection molding.

II. UV Sterilization

An aqueous bacteria suspension was prepared by suspending a capsule of dry bacteria powder containing 5 billion CFU (colony forming units) in 250 ml sterile water. The dry bacteria powder was a mixture of probiotic strains encompassing Lactobacillus acidophilus, Lactobacillus plantarum, Bifidobacterium lactis, Bifidobac bifidum, Lactobacillus rhamnosus, and Bifidobacterium breve. This bacteria suspension was incubated for 1 hour at 20° C.

In each case 10 ml of this bacteria suspension were put into a ceramic beaker with an inner diameter of 6.8 cm and exposed to UV radiation, whereupon the bacteria suspension in the beaker was placed under a UV-C lamp at a constant distance of 6 cm (distance from lamp to surface of suspension). The spectrum of the UV-C lamp ranged from 260 nm to 300 nm, peaking at 275 nm. Intensity at 265 nm was about 20% of peak.

In examples UV1-UV4 plaques (2×3 inches, thickness of 0.125 inch/3.175 mm), which were produced from acrylic-based polymers A3 (comparative example UV1 and UV2) and A4 (examples UV3 and UV4) via injection molding, were placed between the lamp and bacteria suspension. The bacteria suspensions were exposed to the UV radiation for 30 minutes or 60 minutes at 20° C. During UV exposure the bacteria suspensions were gently shaken every 7.5 minutes.

For each experiment, petri dishes with agar growth medium (inner diameter of 90 mm, height of 15 mm) were inoculated with the bacteria suspension after exposure time using a sterile inoculation loop.

Control 1 (C1) was exposed to the UV radiation for 30 minutes at 20° C. without covering the bacteria suspension. During UV-exposure the bacteria suspension was gently shaken every 7.5 minutes. In Control 2 (C2) the bacteria suspension was not exposed to UV radiation and used for inoculation directly after incubation (1 hour, 20° C.).

Afterwards, all petri dishes were incubated at 20° C. for 48 hours.

The bacteria growth is shown in FIG. 1 and summarized in the following table 1. The intensity of the bacterial growth after 48 hours incubation time is ranged from 0-5 (0=no visible growth to 5=nearly closed bacterial lawn).

TABLE 1
Test results of UV sterilization

| | | | Intensity |
|---|---|---|---|
| | | | of |
| | | Observation after 48 h | bacterial |
| Ex. | Description | incubation of petri dishes | growth |
| C1 | Control 1/ | No visible bacterial growth | 0 |
| | 30 min UV exposure, | | |
| | not covered | | |
| C2 | Control 2/ | Visible bacterial growth, | 5 |
| | without UV exposure | nearly closed bacteria lawn | |
| | | of about 2 cm diameter | |
| UV1* | 30 min UV exposure, | Visible bacterial growth, | 4 |
| | covered with A3 plaque | large number of bacterial | |
| | | colonies in an area of about | |
| | | 2 cm diameter, slightly less | |
| | | growth compared with C1 | |
| UV2* | 60 min UV exposure, | Visible bacterial growth, | 3 |
| | covered with A3 plaque | small number of single | |
| | | bacterial colonies in an area | |
| | | of about 2 cm diameter, less | |
| | | bacterial growth compared | |
| | | with C2 | |
| UV3 | 30 min UV exposure, | Visible bacterial growth, | 3 |
| | covered with A4 plaque | small amount of single | |
| | | bacterial colonies in an area | |
| | | of about 1 cm diameter, | |
| | | significant less bacterial | |
| | | growth compared with Ex | |
| | | UV1 | |
| UV4 | 60 min UV exposure, | Slightly visible bacterial | 1 |
| | covered with A4 plaque | growth, very small number | |
| | | of single bacterial colonies in | |
| | | an area of about 1 cm | |
| | | diameter, significant less | |
| | | bacterial growth compared | |
| | | with Ex UV2 | |

*comparative example

Comparative example UV1 (30 minutes exposure) shows slightly less bacterial growth compared with control C2. Inventive example UV3 (30 minutes exposure) shows significant reduction of bacterial growth compared with control C2. Comparing the examples UV1 and UV2 (comparative, covered with A3) with the inventive example UV3 and UV4 (covered with A4) each after 30 minutes or 60 minutes exposure, it is shown that the UV sterilization is significantly more effective using the A4 covering material. UV exposure for 60 minutes can result sufficient sterilization. The sterilization effect could be improved via optimizing of the UV source.

III. UV/VIS Transmission Spectra

Test specimens (plaques of 2×3 inches, thickness of 0.125 inch/3.175 mm) were prepared from the acrylic-based polymer materials A1-A3 via injection molding.

The transmission spectra in the UV/Vis wavelength range from 240 to 900 nm were measured using a Cary 5000 Spectrophotometer in accordance with ISO 134.68-2 at a thickness of about 3 mm (0.125 inch). The transmission spectra are shown in the FIGS. 2 (range 240 to 900 nm) and 3 (range 240 to 400 nm). The averaged transmittance in the wavelength range of 260 to 300 nm are as follows: about 41.6% for A1, about 38.5% for A2 and 0.2% for comparative example A3. The material A4 (transmission spectra not shown) exhibits a transmission spectrum and an averaged transmittance in the wavelength range of 260 to 300 nm which are very similar to A1.

All samples showed a high transparence in the visible range and good optical appearance.

1. A method for UV sterilization of a surface of a formed article, including sterilization of an inner surface of the formed article, the method comprising:
a. providing the formed article, wherein at least one part of said formed article is made from an acrylic-based polymer material, wherein the acrylic-based polymer material comprises at least 50 wt.-%, based on the acrylic-based polymer material, of at least one alkyl (meth)acrylate polymer A; and
b. exposing an outer surface of the formed article to UV radiation at a wavelength in the range of 260 nm to 300 nm;
wherein the acrylic-based polymer material has a transmittance of at least 10%, averaged over the wavelength range from 260 nm to 300 nm, and measured in accordance with ISO 13468-2 at a thickness of 3 mm.

2. The method according to claim 1, wherein the acrylic-based polymer material has a transmittance of at least 10% at a wavelength of 265 nm, measured in accordance with ISO 13468-2 at a thickness of 3 mm.

3. The method according to claim 1, wherein the acrylic-based polymer material comprises, based on the total weight of the acrylic-based polymer material:
A. 50.0 to 100.0 wt.-% of at least one alkyl(meth)acrylate polymer A;
B. 0.0 to 40.0 wt.-% of at least one impact modifier B;
C. 0.0 to 50.0 wt.-% of at least one polymeric component C, different from A and B;
D. 0.0 to 20.0 wt.-% of at least one plasticizer D, and
E. 0.0 to 10.0 wt.-% of at least one further component E.

4. The method according to claim 1, wherein the acrylic-based polymer material comprises:
at least 50 wt.-% based on the acrylic-based polymer material, of at least one alkyl (meth)acrylate polymer A;
at least one impact modifier B selected from particulate multiphase graft copolymers, wherein the at least one impact modifier B comprises an elastomeric phase built up from crosslinked C₁-C₁₀ alkyl acrylate, and a hard outer shell comprising at least one C₁-C₁₀ alkyl (meth)acrylate; and
at least one polymeric component C comprising polyvinylidene difluoride (PVDF),
wherein the ratio of the components A:C is in the range of 3:1 to 4:1.

5. The method according to claim 1, wherein the at least one alkyl(meth)acrylate polymer A comprises, based on the total weight of the at least one alkyl(meth)acrylate polymer A:
70.0 to 100.0 wt.-% of at least one alkyl methacrylate monomer having from 1 to 20 carbon atoms in the alkyl radical, and
0.0 to 30.0 wt.-% of at least one alkyl acrylate monomer, having from 1 to carbon atoms in the alkyl radical.

6. The method according to claim 1, wherein the formed article is an article selected from the group consisting of a medical device, a container used for cosmetic or pharmaceutical products, a container used in the food industry, a package used for cosmetic or pharmaceutical products, and a package used in the food industry.

7. The method according to claim 1, wherein the UV radiation at a wavelength in the range of 260 to 300 nm is provided by one or more UV light emitting diodes (UV-LEDs),
wherein at least a significant amount of emission spectrum of the UV-LEDs is in the range of 260 nm to 300 nm.

8. The method according to claim 1, wherein the exposure of the outer surface of the formed article to UV radiation is carried out in such way that a UV dose in the range of 1 to 10.000 mJ/cm² is effected.

9. The method according to claim 1, wherein the method for UV sterilization inhibits the growth of one or more microorganisms of a genus selected from the group consisting of Escherichia, Salmonella, Listeria, Aspergillus, Bacillus, Cryptosporidium, Clostridium, Streptomyces, Aeromonas, Candida, Helicobacter Klebsiella, Legionella, Listeria, Pseudomonas, Staphylococcus, Streptococcus, Lactobacillus, Bifidobacterium, Oenococcus, and Saccharomycodes.

10. The method according to claim 1, wherein the formed article is surrounded by a protective packaging during UV exposure, wherein the protective packaging is made of an UV-transmissive polymer material.

11. A formed article for use in the method for UV sterilization according to claim 1, wherein at least one part of said formed article is made from an acrylic-based polymer material, wherein the acrylic-based polymer material comprises at least 50 wt.-%, based on the acrylic-based polymer material, of at least one alkyl(meth)acrylate polymer; and
wherein the acrylic-based polymer material has a transmittance of at least 10%, averaged over the wavelength range from 260 nm to 300 nm, and measured in accordance with ISO 13468-2 at a thickness of 3 mm.

12. The formed article according to claim 11, wherein the formed article is an article selected from the group consisting of a medical device, a container used for cosmetic or pharmaceutical products, a container used in the food industry, a package used for cosmetic or pharmaceutical products, and a package used in the food industry.

13. The formed article according to claim 11, wherein the formed article is a medical device selected from the group consisting of a medical diagnostic device, an intravenous and catheter accessory, a blood handling device, a chest drainage unit, a respiratory ventilating device, a medical filter housing, a permanent device housing, a tube, a connector, a fitting, and a cuvette.

14. A process for producing the formed article according to claim 11, the process comprising:
forming the acrylic-based polymer material with a method selected from the group consisting of injection molding, blow molding, and thermoforming.

15. The method according to claim 3, wherein the at least one impact modifier B is a particulate multiphase graft copolymer.

16. The method according to claim 3, wherein the at least one polymeric component C is selected from the group consisting of a polyvinylidene fluoride (PVDF) and a polyethylene glycol (PEG) having a molecular weight of at least 10,000 g/mol.

17. The method according to claim 3, wherein the at least one further component E is selected from the group consisting of an additive, an auxiliary, and a filler.

18. The method according to claim 4, wherein the at least one impact modifier B comprises an elastomeric phase built up from n-butyl acrylate.

19. The method according to claim 4, wherein the at least one impact modifier B comprises a hard outer shell comprising methyl methacrylate.

20. The method according to claim 10, wherein the protective packaging is made of a UV-transmissive polymer material selected from the group consisting of polyethylene, polypropylene, polyvinylchloride, and the acrylic-based polymer material.
